(19) United States
(12) Patent Application Publication
(10) Pub. No.: US 2007/0005042 A1
(43) Pub. Date: Jan. 4, 2007

(54) Programmable Intraosseous Drug Delivery System

(75) Inventor: Rolfe C. Anderson, Saratoga, CA (US)

Correspondence Address: DEWIPAT INCORPORATED, P.O. BOX 1017, CYPRESS, TX, 77410-1017, US

(73) Assignee: ALZA CORPORATION, Mountain View, CA (US)

(21) Appl. No.: 11/427,716

(22) Filed: Jun. 29, 2006

Related U.S. Application Data
(60) Provisional application No. 60/695,618, filed on Jun. 30, 2005.

(51) Int. Cl. A61K 9/22; A61C 5/00

(52) U.S. Cl. 604/890.1; 433/217.1

(57) ABSTRACT

A device for intraosseous drug delivery includes a first implant body having a delivery orifice at a distal end thereof. The first implant body is adapted for mounting in a jawbone such that the delivery orifice communicates with the jawbone. The device further includes a drug cartridge including a reservoir for a drug disposed in the first implant body. The device further includes a pump disposed in the first implant body for pumping the drug from the reservoir to the delivery orifice.

### BACKGROUND OF THE INVENTION

The invention relates generally to methods and apparatus for delivering drugs or therapeutic agents.

Treatments for those who suffer from diseases and/or illnesses such as diabetes, Parkinson's disease, Hepatitis C, epilepsy, hypertension, congestive heart failure (CHF), muscular sclerosis (MS), and chronic pain rely on systematic drug administration. There are various routes of administering drugs. For example, drugs may be injected intravenously or intramuscularly, which lead directly into a patient's bloodstream. Alternatively, drugs may be absorbed through mucous membrane (or linings) of the ocular, nasal, vaginal, rectal, or oral cavity. Each of these routes of administration have their respective benefits over peroral administration, insofar as these routes bypass the first-pass effect and avoid the pre-systemic elimination within the gastrointestinal tract. However, these alternative routes of administration have their limitations. For example, intra-rectal and intra-vaginal can be inconvenient and uncomfortable, and the latter is not available to the entire population. On the other hand, intra-nasal delivery typically requires use of potentially toxic “penetration enhancers” to effect passage of the drug across the nasal and mucosa, which is characterized by a thick layer that is resistant to the passage of macromolecules. Injection (intravenous or intramuscular) tends to be undesirable in a number of respects. First, many patients find it difficult and burdensome to inject themselves as frequently as required. Such reluctance can lead to non-compliance, which in the most serious cases can be life-threatening. Additionally, repeated injection at a single location on the body results in lumps or small dents, called “lipodystrophies.” The oral cavity, on the other hand, is generally considered a convenient and comfortable site of administration.

International Application Publication No. WO 2004/069076 A2 (Wolff et al.) discloses drug delivery devices for implantation in an oral cavity that delivers a drug in a controlled and programmable manner. The drug delivery device may be built into a prosthetic tooth crown, a denture plate, braces, or a dental implant. Wolff et al. disclose both a passively controlled drug delivery device, which relies on a dosage form, and an electro-mechanically controlled drug delivery device for secreting, releasing, and otherwise delivering a drug into a patient's mouth. In Wolff et al., the drug delivery device is adapted for drug absorption by buccal (i.e., placing a drug between the gums and the cheek), sublingual (i.e., placing a drug under the tongue), labial mucosa, and/or soft-palatal drug absorption. Wolff et al. note that chewing, sucking, as well as buccal and sublingual administration leads to direct absorption via the oral cavity, which is a route that avoids the pre-systemic elimination within the gastrointestinal tract and the first-pass metabolism in the liver, as previously mentioned.

However, reliance on buccal and sublingual, labial mucosa, and/or soft-palatal drug absorption also has potential limitations. For example, sublingual mucosa is more permeable than the buccal mucosa; however, the sublingual mucosa lacks an expanse of smooth muscle or immobile mucosa. Furthermore, the sublingual mucosa is constantly being washed by a considerable amount of saliva. Therefore, the sublingual mucosa is not ideal for systematic drug administration. On the other hand, the buccal mucosa provides a more reliable route for routine drug delivery. However, the buccal mucosa is less permeable and thus is not able to give a rapid onset of drug absorption. Therefore, buccal mucosal delivery suffers from low flux which, consequently, leads to low drug bioavailability. Further, buccal mucosal delivery lacks dosage retention at the site of absorption.

In general, drug absorption via mucus membrane of the oral cavity is subject to salivary dilution of the drug, accidental swallowing, and inability to localize the drug solution within a specific site of the oral cavity. Other limitations of oral drug absorption include ensuring the drug formulation has an agreeable taste (which can be challenging), not to mention molecular weight limits and potential of variability of dosing with respect to permeability. Therefore, oral drug absorption faces particular challenges with certain drugs (e.g., insulin, and levadopa), which require strict monitoring, precise dosing, and periodic adjustments to dosing in view of the monitoring.

From the foregoing, there is desired a practical method and drug delivery apparatus for controlled, programmable administration of drugs that takes advantage of drug administration in the oral cavity, but overcomes the limitations of oral drug absorption.

### SUMMARY OF THE INVENTION

In one aspect, the invention relates to a device for intraosseous drug delivery which comprises a first implant body having a delivery orifice at a distal end thereof, the first implant body adapted for mounting in a jawbone such that the delivery orifice communicates with the jawbone, a drug cartridge including a reservoir for a drug disposed in the first implant body, a pump disposed in the first implant body for pumping the drug from the reservoir to the delivery orifice, and a communications line through which the pump can receive power and control signals.

In another aspect, the invention relates to a system for intraosseous drug delivery which comprises a first implant body having a delivery orifice at a distal end thereof, the first implant body adapted for mounting in a jawbone such that the delivery orifice communicates with the jawbone, a drug cartridge including a reservoir for a drug disposed in the first implant body, and a pump disposed in the first implant body for pumping the drug from the reservoir to the delivery orifice, a second implant body adapted for mounting in the jawbone, the second implant body containing a control circuit module, and a communications line coupling the control circuit module to the pump such that the pump receives control signals from the control circuit module to deliver the drug to the delivery orifice at a desired rate.

In yet another aspect, the invention relates to a method for intraosseous drug delivery which comprises transmitting signals to a pump in a first implant body mounted in a jawbone from a control circuit module in a second implant body mounted in the jawbone, pumping a drug from a reservoir in the first implant body to a delivery orifice at a distal end of the first implant body in response to the signals, and delivering the drug from the delivery office to the jawbone.

Other features and advantages of the invention will be apparent from the following description and the appended claims.

### BRIEF DESCRIPTION OF THE DRAWINGS

FIG. 1 shows a programmed intraosseous drug delivery system according to one embodiment of the invention embedded in a jawbone.

FIG. 2 is a cross-sectional view of a drug device according to one embodiment of the invention.

FIG. 3 is a cross-sectional view of a controller device according to one embodiment of the invention.

FIG. 4 shows the drug device and controller device coupled together to provide intraosseous drug delivery.

### DETAILED DESCRIPTION OF THE INVENTION

The invention will now be described in detail with reference to a few preferred embodiments, as illustrated in accompanying drawings. In the following description, numerous specific details are set forth in order to provide a thorough understanding of the invention. It will be apparent, however, to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow.

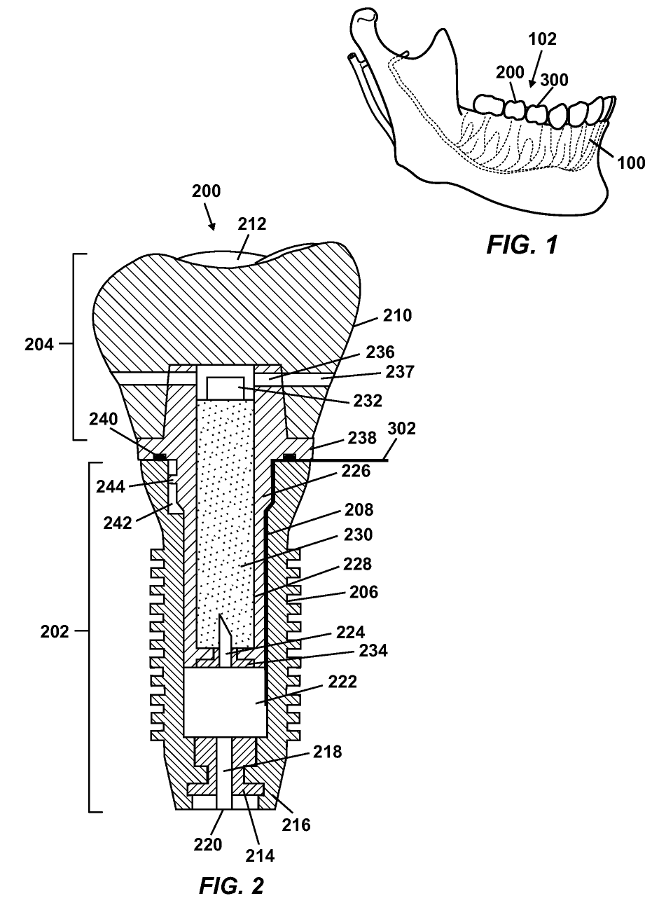

FIG. 1 illustrates a jawbone 100 in which a programmed intraosseous drug delivery system 102 according to an embodiment of the invention is embedded. The programmed intraosseous drug delivery system 102 includes a drug device 200 and a controller device 300. The drug device 200 and controller device 300 are prosthetic tooth devices adapted for surgical implantation into the jawbone 100. The drug device 200 and controller device 300 are in communication. The communications line may be wired or wireless. The drug device 200 stores a drug and includes a delivery orifice in communication with the jawbone 100 and a pump for pumping the drug through the delivery orifice into the jawbone 100. The drug pumped into the jawbone 100 may be absorbed into the vascular system, thereby reducing or obviating the invasive practice of subcutaneous injection for controlled or patterned drug delivery. The controller device 300 controls and regulates pumping of the drug from the drug device 200 into the jawbone 100.

FIG. 2 shows a cross-sectional view of the drug device 200. The drug device 200 has a root portion 202 and crown portion 204. The root portion 202 anchors the drug device in the jawbone (100 in FIG. 1). The root portion 202 includes an implant body 206 which defines a receptacle for a drug cartridge 208. The crown portion 204 includes a crown body 210. The crown body 210 retains the drug cartridge 208 in the implant body 206. The crown body 210 has a chewing surface 212, which allows the drug device 200 to function as a normal tooth. The crown body 210 may be secured to the implant body 206 by a variety of methods, e.g., via a spring latch, a set screw, an adhesive, or a magnetic latch. Preferably, the crown portion 210 is secured to the implant body 206 such that it is removable from the implant body 206 as desired to allow access to the drug cartridge 208.

The implant body 206 is a hollow structure. A needle base 214 is mounted at the base 216 of the implant body 206. The needle base 214 could be molded into the base 216 of the implant body 206. Alternatively, a seat may be formed in the base 216 for receiving the needle base 214. The needle base 214 holds a needle 218. This needle 218 provides a delivery orifice 220 at the base 216 of the implant body 206 through which drug from the drug cartridge 208 can be delivered to the jawbone (100 in FIG. 1). Hereafter, the needle 218 would be referred to as the outlet needle. A pump 222 is mounted on the needle base 214. In this position, the outlet needle 218 forms a passage between the pump 222 and the delivery orifice 220. A needle 224 is provided on top of the pump 222. The needle 224 allows fluid communication between the drug cartridge 208 and the pump 222. Hereafter, the needle 224 would be referred to as the inlet needle. The pump 222 may be an electromechanical pump.

The drug cartridge 208 has a cartridge body 226 which defines a reservoir 228 for holding a quantity of a drug formulation 230. Examples of drugs that may be delivered using the drug device 200 include, but are not limited to, risperidone, hydromorphone, interferon, remicaid, insulin, and erythropoietin. The drug formulation 230 must be in flowable form to enable delivery by the pump 222. A piston 232 is disposed in the cartridge body 226, above the reservoir 228. The position of the piston 232 in the cartridge body 226 changes as the level of drug formulation 230 in the reservoir 228 changes. The position of the piston 232 may be monitored to determine when the drug cartridge 208 should be replaced. A septum 234 is provided at the base of the cartridge body 226 to prevent seepage of the drug from the reservoir 228 before the drug cartridge 208 is mounted on the pump 222. The septum 234 is pierced by the inlet needle 224 when the cartridge body 226 is mounted on the pump 222. An alternative to using the septum 234 is to provide the drug formulation 230 in a collapsible bladder, which would serve as the reservoir 228 and would be pierced by the inlet needle 224 when the cartridge body 226 is mounted on the pump 222.

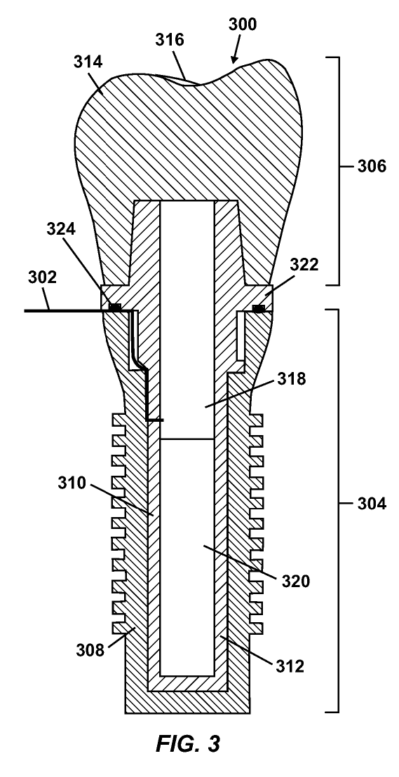

The pump 222 draws the drug formulation 230 from the reservoir 228 through the inlet needle 224 and discharges the drug formulation 230 into the jawbone (100 in FIG. 1) through the outlet needle 218. The pump 222 receives command and power signals from the controller device (300 in FIG. 3) through a communications line, such as a multi-lead cable 302. The cartridge body 226 includes relief ports 236 which are aligned with ports 237 in the crown body 210, thereby allowing fluid from the oral cavity to enter the cartridge body 226 and fill the space created above the reservoir 228 as the level of the drug formulation 230 in the reservoir 228 drops. The piston 232 may extend to the wall of the cartridge body 226 and form a barrier between the fluid entering the cartridge body 226 from the oral cavity and the drug formulation 230 if desired. The cartridge body 226 includes a flange 238 which rests on the upper end of the implant body 206 when the drug cartridge 208 is inserted in the implant body 206. A seal 240, such as an O-ring seal, is typically provided to seal between the flange 238 and the upper end of the implant body 206. The seal 240 may also prevent unintended sliding motion between the contacting surfaces of the flange 238 and implant body 206. The implant body 206 may include slots 242 configured to receive tabs 244 on the drug cartridge 208, thereby facilitating positioning of the drug cartridge 208 in the implant body 206. The slots 242 may interlock with the tabs 244 to secure the drug cartridge 208 to the implant body 206.

FIG. 3 shows a cross-sectional view of the controller device 300 which regulates the pump (222 in FIG. 2) such that the drug formulation (230 in FIG. 2) is delivered to the jawbone (100 in FIG. 1) at a desired rate or pattern. The controller device 300 includes a root portion 304 and a crown portion 306. The root portion 304 anchors the controller device 300 in the jawbone (100 in FIG. 1). The root portion 304 includes an implant body 308 which defines a receptacle for a controller cartridge 310. The crown portion 306 includes a crown body 314. The crown body 314 retains the controller cartridge 310 in the implant body 308. The crown body 314 has a chewing surface 316, which allows the controller device 300 to function as a normal tooth. The crown body 314 may be secured to the implant body 308 by a variety of methods, e.g., via a spring latch, a set screw, an adhesive, or a magnetic latch. Preferably, the crown body 314 is secured to the implant body 308 such that it is removable from the implant body 308 as desired to allow access to the controller cartridge 310.

The controller cartridge 310 has a cartridge body 312. A flange 322 on the cartridge body 312 rests on an upper end of the implant body 308 when the controller cartridge 310 is inserted in the implant body 308. A seal 324, such as an 0-ring seal, seals between the flange 322 and the upper end of the implant body 308. The seal 324 may also prevent unintended sliding motion between the contacting surfaces of the flange 322 and implant body 308. The cartridge body 312 receives a control circuit module 318 and a power module 320. The power module 320 may include one or more batteries. The control circuit module 318 includes electronics for controlling operation of the pump (222 in FIG. 2). The control circuit module 318 is electrically coupled to the power module 320 and receives power from the power module 320. Control and power signals are sent from the control circuit module 318 to the pump (222 in FIG. 2) through the multi-lead cable 302. The control circuit module 318 may include an internal antenna for external communication, e.g., to receive commands from an external control system. The implant body 308 may also serve as a secondary antenna.

The control circuit module 318 may receive input from one or more sensors (not shown) which respond to a physiological attribute or delivery conditions in the drug device (200 in FIG. 2). The sensor(s) may be disposed within the drug device (200 in FIG. 2) or in another location inside or outside of the body, e.g., under or on the skin. Examples of physiological attributes that may be monitored include, but are not limited to, interstitial-fluid drug concentration level, interstitial-fluid glucose level, tissue temperature, blood pressure, and heart rate. Where the sensor(s) is located remote to the controller device 300, the control circuit module 318 may communicate with the sensor(s) using a variety of methods, for example, ultrasound, IR, and RF. The remote sensor(s) may communicate continuously, at intervals, in reply to interrogation, or in the event of a sudden change in a measured physiological attribute.

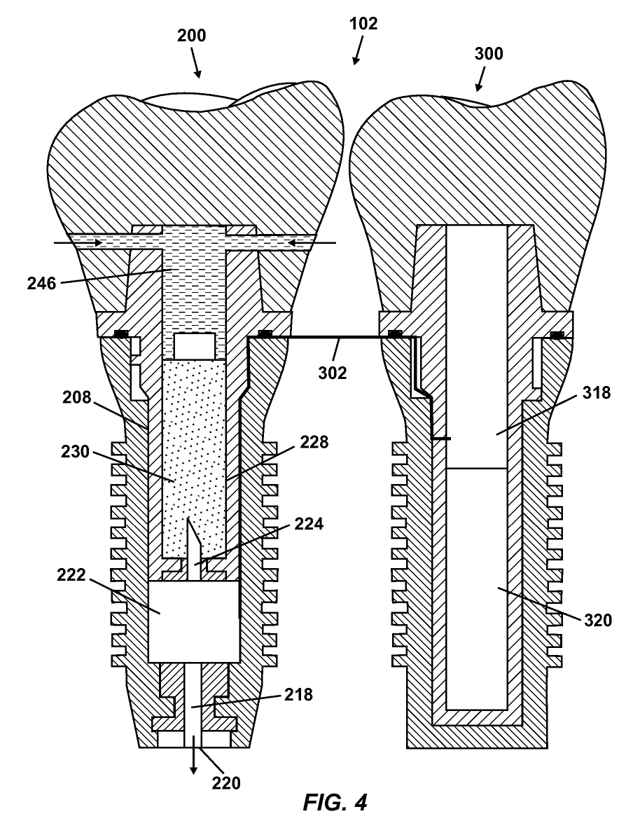

FIG. 4 shows the programmed intraosseous drug delivery system 102 including the drug device 200 coupled to the controller device 300 via the multi-lead cable 302. The drug device 200 and controller device 300 are installed in a jawbone (100 in FIG. 1) by first extracting two teeth from the jawbone. The extracted teeth may or may not be next to each other. The drug device 200 is installed in the jawbone such that drug from the drug device 200 can be delivered to the jawbone through the delivery orifice 220. In operation, the pump 222 receives control and power signals from the control circuit module 318. In response to the control signals, the pump 222 draws the drug formulation 230 from the reservoir 228 through the inlet needle 224 and discharges the drug formulation 230 into the outlet needle 218, wherein the drug formulation 230 is discharged through the delivery orifice 220 into the jawbone. As the level of the drug formulation 230 drops, fluid 246 from the oral cavity enters the drug cartridge 208 to relieve the vacuum created in the drug cartridge 208.

While dispensing the drug formulation 230, the control circuit module 318 may transmit signals to a computer system (not shown) to provide real-time monitoring of a patient's dosing. Once most of or the entire drug formulation 230 has been expelled from the drug cartridge 208 or after expiration of a certain time period, the drug cartridge 208 may be replaced by a patient or a caregiver. The invention allows administration of a specified dosage of a drug at a specified and adjustable delivery rate or pattern.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A device for intraosseous drug delivery, comprising:
a first implant body having a delivery orifice at a distal end thereof, the first implant body adapted for mounting in a jawbone such that the delivery orifice communicates with the jawbone; and
a drug cartridge including a reservoir for a drug disposed in the first implant body;
a pump disposed in the first implant body for pumping the drug from the reservoir to the delivery orifice; and
a communications line through which the pump can receive power and control signals.

2. The device of claim 1, further comprising a crown body secured to the first implant body.

3. The device of claim 2, wherein the drug cartridge includes one or more ports which allow a fluid to enter the drug cartridge.

4. The device of claim 3, wherein the crown body includes one or more ports aligned with the one or more ports in the drug cartridge, the one or more ports in the crown body providing communication between the one or more ports in the drug cartridge and an exterior of the crown body.

5. The device of claim 3, further comprising a piston disposed in the drug cartridge, wherein the piston moves in the drug cartridge as the level of drug in the reservoir changes.

6. The device of claim 1, wherein a needle disposed at a base of the first implant body provides the delivery orifice.

7. The device of claim 1, wherein the pump communicates with the reservoir through a needle and septum.

8. The device of claim 7, wherein the septum is mounted at a base of the drug cartridge, and the needle is mounted on the pump.

9. The device of claim 1, wherein the pump is an electromechanical pump.

10. The device of claim 1, wherein a seal is provided between the drug cartridge and the first implant body.

11. The device of claim 1, further comprising a second implant body adapted for mounting in the jawbone, the second implant body containing a control circuit module which is coupled to the pump through the communications line, wherein the control circuit module controls operation of the pump such that the drug is delivered to the delivery orifice at a desired rate.

12. The device of claim 11, further comprising a crown body secured to the second implant body.

13. The device of claim 11, wherein the second implant body further comprises a power module coupled to the control circuit module, and the control circuit module is configured to deliver the power and control signals to the pump.

14. The device of claim 11, wherein the control circuit module includes an internal antenna for external communication.

15. The device of claim 11, wherein the communications line is provided by a multi-lead cable.

16. A system for intraosseous drug delivery, comprising:
a first implant body having a delivery orifice at a distal end thereof, the first implant body adapted for mounting in a jawbone such that the delivery orifice communicates with the jawbone;
a drug cartridge including a reservoir for a drug disposed in the first implant body;
a pump disposed in the first implant body for pumping the drug from the reservoir to the delivery orifice;
a second implant body adapted for mounting in the jawbone, the second implant body containing a control circuit module; and
a communications line coupling the control circuit module to the pump such that the pump receives control signals from the control circuit module to deliver the drug to the delivery orifice at a desired rate.

17. The system of claim 16, further comprising a power module disposed in the second implant body, the power module being electrically coupled to the control circuit module.

18. A method for intraosseous drug delivery, comprising:
transmitting signals to a pump in a first implant body mounted in a jawbone from a control circuit module in a second implant body mounted in the jawbone;
pumping a drug from a reservoir in the first implant body to a delivery orifice at a distal end of the first implant body in response to the signals; and
delivering the drug from the delivery orifice to the jawbone.
